(19) United States
(12) Patent Application Publication
(10) Pub. No.: US 2024/0366750 A1
(43) Pub. Date: Nov. 7, 2024

(54) VIRAL ANTIGEN DISPLAY COMPOSITIONS AND METHODS

(71) Applicant: REGENTS OF THE UNIVERSITY OF MINNESOTA, Minneapolis, MN (US)

(72) Inventor: Alon M. Herschhorn, Minneapolis, MN (US)

(21) Appl. No.: 18/654,961

(22) Filed: May 3, 2024

Related U.S. Application Data
(60) Provisional application No. 63/464,358, filed on May 5, 2023.

(51) Int. Cl. A61K 39/21; A61K 9/127; A61K 9/51; A61K 39/00; A61P 31/14; C12N 7/00

(52) U.S. Cl. CPC A61K 39/21; A61K 9/127; A61K 9/5123; A61P 31/14; C12N 7/00; A61K 2039/55555; C12N 2740/16023; C12N 2740/16051; C12N 2740/16234

(57) ABSTRACT

A composition generally includes a virus-like particle (VLP) and a truncated form of a viral envelope protein displayed on the VLP. Generally, the truncated form of the envelope protein has at least a portion of the cytoplasmic tail region of the envelope protein deleted. The VLP may be generated by transfection with a first polynucleotide that encodes a VLP subunit protein and second polynucleotide that encodes the truncated envelope protein. The transfection may be performed in vitro or in vivo.

### CROSS-REFERENCE TO RELATED APPLICATION

This application claims the benefit of U.S. Provisional Patent Application No. 63/464,358, filed May 5, 2023, which is incorporated herein by reference in its entirety.

### GOVERNMENT FUNDING

This invention was made with government support under DA049279 awarded by the National Institutes of Health. The government has certain rights in the invention.

### SEQUENCE LISTING

This application contains a Sequence Listing electronically submitted via Patent Center to the United States Patent and Trademark Office as an .xml file entitled “0110-000723US01.xml” having a size of 4,961 bytes and created on May 2, 2024. The information contained in the Sequence Listing is incorporated by reference herein.

### SUMMARY

This disclosure describes, in one aspect, a composition that generally includes a virus-like particle (VLP) and a truncated form of a viral envelope protein displayed on the VLP. Generally, the truncated form of the envelope protein has at least a portion of the cytoplasmic tail region of the envelope protein deleted. In one or more embodiments, the entire cytoplasmic tail region of the envelope protein may be deleted.

In one or more embodiments, the VLP includes HIV-1 Gag and the truncated form of the viral envelope protein comprises HIV-1 envelope protein with at least a portion of the cytoplasmic tail deleted. In one or more embodiments, the truncated form of the HIV-1 envelope protein is amino acids 1-709 of SEQ ID NO: 1.

In one or more embodiments, the composition can further include an adjuvant.

In another aspect, this disclosure describes a composition that generally includes a first polynucleotide encoding a VLP subunit protein, a second polynucleotide encoding a viral envelope protein comprising a deletion of at least a portion of a cytoplasmic tail, and a delivery vehicle attached to or encapsulating the first polynucleotide and the second polynucleotide. In one or more embodiments, the delivery vehicle may be a lipid nanoparticle.

In another aspect, this disclosure describes a composition that generally includes a first polynucleotide attached to or encapsulated by a first delivery vehicle, the first polynucleotide encoding a VLP subunit protein, and a second polynucleotide attached to or encapsulated by a second delivery vehicle, the second polynucleotide encoding a viral envelope protein comprising a deletion of at least a portion of a cytoplasmic tail. In one or more embodiments, the first delivery vehicle, the second delivery vehicle, or both delivery vehicles may be a lipid nanoparticle.

In another aspect, this disclosure describes a method of preparing a VLP-based vaccine. Generally, the method includes transfecting a cell with a first polynucleotide encoding a VLP subunit protein, transfecting the cell with a second polynucleotide encoding a truncated form of a viral envelope protein, allowing the cell to express the first polynucleotide to produce VLP subunits, allowing the cell to express the second polynucleotide to produce the truncated viral envelope protein, and allowing the VLP subunits and the truncated viral envelope protein to assemble into a VLP in which the truncated viral envelope protein is displayed on the surface of the VLP.

In one or more embodiments, the cell is transfected in vitro.

In one or more embodiments, the cell is transfected in vivo.

In one or more embodiments, the cell is transfected with the first polynucleotide and the second polynucleotide simultaneously.

In one or more embodiments, the cell is transfected with the first polynucleotide and the second polynucleotide separately.

In one or more embodiments, transfecting the cell with the first polynucleotide or transfecting the cell with a second polynucleotide comprises using a lipid nanoparticle delivery vehicle.

In another aspect, this disclosure describes a method of treating a subject having, or at risk of having, an infection by an enveloped virus. Generally, the method includes administering a composition to the subject in an amount effective to treat infection by the enveloped virus. Generally, the composition includes a virus-like particle (VLP) and a truncated form of a viral envelope protein displayed on the VLP.

In yet another aspect, this disclosure describes a method of treating a subject having, or at risk of having, an infection by an enveloped virus. Generally, the method includes administering a composition to the subject in an amount effective to treat infection by the enveloped virus. Generally, the composition includes a first polynucleotide encoding a VLP subunit protein, a second polynucleotide encoding a truncated form of a viral envelope protein, and one or more delivery vehicles attached to or encapsulating the first polynucleotide and the second polynucleotide. In one or more embodiments, the first polynucleotide and the second polynucleotide may be attached to or encapsulated by a single delivery vehicle. In one or more embodiments, the first polynucleotide may be attached to or encapsulated by a first delivery vehicle and the second polynucleotide may be attached to or encapsulated by a second delivery vehicle.

In one or more embodiments, the delivery vehicle comprises a lipid nanoparticle.

The above summary is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

FIG. 1. Preparation of mRNA-based lipid nanoparticles (mRNA-LNPs). A scheme for preparation of pseudoviruses (PVs) using purified mRNA or mRNA-LNP for expression of HIV-1 Envs.

FIG. 2. Validation of mRNA-based lipid nanoparticles (mRNA-LNPs) for delivery of HIV-1 vaccine candidates. (A) Infection of Cf2T4R5 target cells by PVs prepared with in vitro transcribed and purified mRNA for HIV-1 Env expression. U*, pseudouridine; IF4, an aptamer that binds eukaryotic translation initiation factor 4G; TEV, sequence derived from the 5′ UTR of the tobacco etch virus. (B) Similar to (A) but mRNA-LNP was used to prepare PVs instead of purified mRNA and the PV infectivity was plotted against increasing amounts of mRNA-LNP used. (C) mRNA-mediated co-expression of HIV-1_{AD8} ΔCT Envs and HIV-1 Gag in 293T cells produces virus-like particles. Western blot of concentrated supernatant-containing VLPs and of mRNA-transfected cells. Numbers indicate mRNA ratio.

FIG. 3. Validation of synthetic virus-like particles (syn-VLPs) for delivery of HIV-1 vaccine candidates. (A) Optimization of syn-VLPs preparation. Different ratios of 1059-SOSIP-Spytag and SpyCatcherVLP were mixed and incubated overnight following by SDS-PAGE analysis to determine optimal conjugation ratio. The syn VLP-spy catcher shape was taken from Brune et al., 2016, Sci. Rep. 6:19234. (B) ELISA of binding of 0.625 μg/ml of different antibodies to 1059-spytag trimer. (C) SDS-PAGE of 1059-SOSIP-Spytag and SpyCatcherVLP conjugated at 6:1 ratio and purified by size exclusion chromatography. (D) Electron microscopy images of assembled 1059-SOSIP-SpyCatcher VLPs that were negatively stained.

FIG. 4. Immunization scheme of six rabbits with conformation-specific immunogens.

FIG. 5. Elicitation of high-titer binding antibodies to conformation-specific HIV-1 Env immunogens delivered by mRNA-LNP and syn-VLPs. (A) Binding of IgG-containing sera from rabbit 1 (Rb1) to soluble HIV-1₁₀₅₉ SOSIP Envs. Sera were collected at specified timepoints, and IgG binding was measured by ELISA. (B) Binding titer of sera from all six rabbits to 1059-SOSIP at indicated time points after immunization. (C) Binding titer of sera from all six rabbits to 1059 gp120 at indicated time points after immunization. (D) Binding titer of sera from all six rabbits to different homologous and heterologous Envs at indicated time points after immunization. NAB9 SOSIP was not well expressed, and therefore the NAB9_{Q658V} mutant that contains a trimeric stabilization mutation (Rawi, R. et al Cell Reports 33, 108432, 2020) was used. (E) Statistical analysis of the differences between the binding titer of group 1 and group 2 rabbits to different HIV-1 Envs.

FIG. 6. Elicitation of high-titer binding antibodies to conformation-specific HIV-1 Env immunogens delivered by mRNA-LNP and syn-VLPs. (A) Average numbers of antibody secreting cells (ASC) in the blood of each rabbit. (B) Comparison of the number of ASC in the 2 groups of rabbits. PI, pre-immune.

FIG. 7. Cellular and neutralizing antibody responses to conformation-specific HIV-1 Env immunogens delivered by mRNA-LNP and syn-VLPs to rabbits. (A) Response of PBMCs (collected 42 weeks post priming) to incubation with HIV-1 Env peptide pool was detected by ELISpot assay. Four measurements (control) or the mean±sem are shown for each rabbit. Results of rabbit 6 PBMCs are from only two measurements due to limited cells availability. Controls are PBMCs incubated with medium without peptide pools. CTRL, positive control of pooled PBMCs stimulated with PMA/Ionomycin (two measurements). (B) Statistical analysis of the difference between the number of PBMCs secreting IFN-γ in response to HIV-1 Env peptides (from panel A) in the two groups of rabbits. Group 1 was boosted (1st boost) with mRNA-LNPs for AD8ΔCT Env expression and Group 2 was boosted with mRNA-LNPs for co-expression of AD8ΔCT Envs and HIV-1 Gag, potentially leading to presentation of ΔCT Envs on virus-like particles in vivo. Color code is similar to panel (A) and represents four measurements (two measurements for R6 and control) for PBMCs from each rabbit.

FIG. 8. Cellular and neutralizing antibody responses to conformation-specific HIV-1 Env immunogens delivered by mRNA-LNP and syn-VLPs to rabbits. (A) Neutralization activity of sera from the six rabbits against viruses pseudotyped with HIV-1₁₀₁₂ Envs. (B) Neutralization titer (Inhibitory dilution 50; ID₅₀) of rabbit sera from week 36 post immunization against 13 T/F strains and against the easy-to-neutralize HIV-1_{SF162} strain (pseudoviruses). ID₅₀ values are color-coded according to the color scale shown on the right. (C) Statistical analysis of the difference between neutralization titer of the two groups of rabbits (FIG. 4). Neutralization results are representative of between 1-3 independent experiments, each performed in duplicate.

FIG. 9. A model for triggers of enhanced neutralization response in rabbits. Immunization with mRNA for co-expressing AD8ΔCT Envs and HIV-1 Gag, which potentially leads to production of VLPs displaying the AD8ΔCT Envs in vivo, can prime diverse T and B cells. Some of these cells can be then activated by immunization with soluble 1059-SOSIP. The overall result is enhanced neutralization and cellular response. ΔCT, cytoplasmic tail-deleted.

FIG. 10. In-vitro transcription (IVT) vectors. IVT plasmids were designed and built to contain bacteriophage T7 RNA polymerase promoter, 5′ untranslated region (UTR), gene of interest, 3′ UTR, and 120-base long polyA tail. IF4, 40-base long aptamer that binds eukaryotic translation initiation factor 4G (Tusup et al., 2019, CHIMIA (Aarau) 73:391-394); TEV, sequence derived from the 5′ UTR of the tobacco etch virus (Gallie et al., 1995, Gene 165:233-238); AES/mtRNR1, 3′ UTR stabilizing elements (Orlandini von Niessen et al., 2019, Molecular Therapy 27:824-836); xb_glo, a sequence derived from the 3′UTR of Xenopus beta-globin gene. GOI, gene of interest.

FIG. 11. Immunization scheme of three groups of seven humanized DRAGA mice with HIV-1 Env immunogens.

FIG. 12. Representative flow cytometry plots reflecting binding of serum from an immunized mouse to HIV-1 envelope glycoproteins.

FIG. 13. Neutralization activity of sera from vaccinated and control humanized DRAGA mice against viruses pseudotyped with HIV-1_{AD8} Envs or HIV-1₁₀₅₉ Envs.

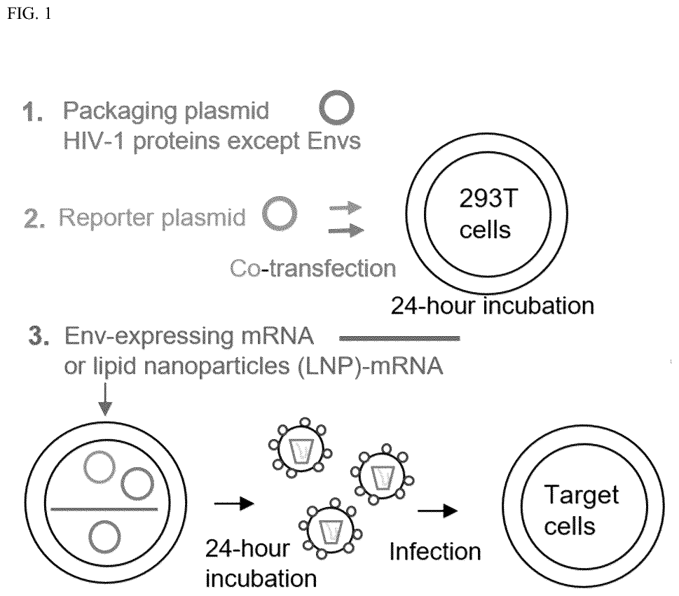

### DETAILED DESCRIPTION OF ILLUSTRATIVE EMBODIMENTS

This disclosure describes a method for displaying viral immunogens on virus-like particles (VLPs). In one exemplary application, the VLPs may be used to elicit neutralizing antibodies against the viral immunogen, which may produce a therapeutic composition for treating infection by the virus that naturally expresses the immunogen. In one or more embodiments, the VLPs display a truncated form of a viral envelope protein in which at least a portion of the cytoplasmic tail of the envelope protein is deleted.

This disclosure further describes an exemplary embodiment in which the method is used to produce a vaccine against HIV-1. In this exemplary embodiment, the VLPs display a truncated form of the HIV-1 envelope protein (amino acids 1-709 of SEQ ID NO:1). While described herein in the context of an exemplary embodiment in which the viral immunogen is an HIV-1 envelope, the method may be practiced using any suitable envelope protein obtained from any virus of interest.

HIV-1 envelope glycoproteins (Envs) mediate viral entry into target cells and are the sole target of neutralizing antibodies. HIV-1 Envs are assembled into a trimeric spike that includes three gp120 exterior subunits associated noncovalently with three gp41 transmembrane subunits. Each Env subunit mediates specific activity: the surface gp120 subunit binds the host CD4 receptor and CCR5/CXCR4 coreceptors, and the transmembrane gp41 facilitates membrane fusion. Either spontaneously or in response to receptor binding, HIV-1 Envs can transit from a metastable, high-potential energy closed conformation (State 1) to downstream states (States 2 and State 3) that are associated with multiple structural rearrangements. Neutralizing antibodies (bnAbs) against HIV-1 Envs pinpoint target sites of Env vulnerability. Although direct and indirect mechanisms of HIV-1 escape from some of the bnAbs in vivo exist, bnAbs still provide promising guidance to the type and properties of elicited antibodies that may inhibit infection to a clinically relevant degree.

Anti-HIV-1 bnAbs can be classified according to their preference to recognize specific Env conformations. Most bnAbs that target the CD4 binding site (CD4bs) as well as those targeting the V1/V2 loop of gp120 prefer to neutralize the Env closed conformation (State 1). These bnAbs still neutralize lab-adapted HIV-1 Envs, which are considered fully open. However, these bnAbs lose antiviral activity against primary HIV-1 Envs of all major clades that are stabilized in downstream conformations by genetic modifications of residues that naturally restrain HIV-1 Env closed conformation. In contrast, bnAbs targeting the membrane proximal external region (MPER) of gp41 preferentially recognize downstream Env conformations (State 2 and State 3), probably because their epitope, which is located near the viral membrane, is more accessible on the open Env conformations. In addition to these two groups, some bnAbs can recognize and neutralize multiple Env conformations.

Simultaneously presenting multiple Env conformations to the immune system may elicit diverse antibodies that preferentially recognize different conformations and/or elicit broad antibodies that recognize multiple Env conformations. Such presentation could potentially address the heterogeneity of Env closed conformation. Many HIV-1 Envs of primary strains, including transmitted/founder (T/F) HIV-1 strains that can establish HIV-1 infection in vivo, prefer to be tightly closed but some of these Envs are incompletely closed and they slightly or transiently expose internal epitopes.

The method disclosed herein generally involves displaying one or more immunogens on a VLP. In one or more embodiments, the VLPs may be produced by transfecting a cell with RNA that encode the VLP components. For example, a cell may be transfected with an mRNA that encodes proteins of the VLP and an mRNA that encodes the protein to be displayed by the VLP. In one or more embodiments, the cell may be transfected using lipid nanoparticles, but the method may be practiced using any suitable transformation method. The cell may be transfected in vitro or in vivo. For in vitro transfection, the cell may be transfected, then incubated under conditions effective to allow the transfected cell to produce VLPs. VLPs secreted into the culture medium may be isolated using any suitable method. For in vivo transfection, the RNA may be administered by any suitable method including, but not limited to, delivery using lipid nanoparticles.

In one exemplary embodiment, HIV-1 envelope glycoproteins (Envs) with a truncated cytoplasmic tail (ΔCT) such as, for example, amino acids 1-709 of SEQ ID NO: 1 were administered as mRNA encapsulated together with mRNA for expression of HIV-1 Gag as lipid nanoparticles to be displayed on viral-like particles (VLPs) in vivo. While described herein in the context of an exemplary embodiment in which the ΔCT-Env protein has the entirety of amino acids 710-854 of SEQ ID NO: 1 deleted, the compositions and methods described herein can involve alternate ΔCT-Env proteins in which a portion, but not all, of amino acids 710-854 of SEQ ID NO: 1 are present. Specific highly expressed Envs (strain HIV-1 AD8) were selected for display and the ΔCT version increases high levels of expression on cell surface.

The ΔCT-Env protein may be displayed on any suitable VLP. While described below in the context of exemplary embodiments in which the VLPs are HIV Gag VLPs produced by mRNA-LNPs (Hou et al., 2021, Nat Rev Mater 6:1078-1094; Zhang et al., 2021, Nat Med 27:2234-2245)—the constructs and methods described herein can involve using any suitable enveloped VLP platform—i.e., and VLP that includes a phospholipid bilayer acquired from an infected cell during virus budding.

The virus-like particle (VLP) can include any particle that includes viral protein assembled to structurally resemble the virus from which they are derived but lack enough of the viral genome so that they are non-replicative and, therefore, noninfectious. A VLP may, therefore, include at least some of the viral genome, but the viral genome is genetically modified so that the viral genes responsible for infectivity and replication are inactivated. Exemplary VLPs include, but are not limited to, those based on the capsid and core proteins of any enveloped viruses such as Hepatitis B virus, measles virus, Sindbis virus, the retroviral Gag protein, animal hepadnavirus core Antigen VLPs, and insect viruses.

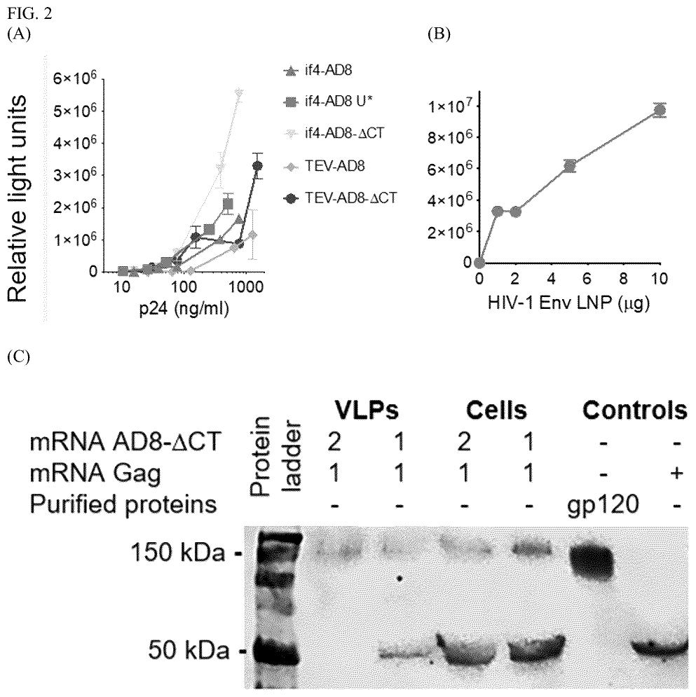

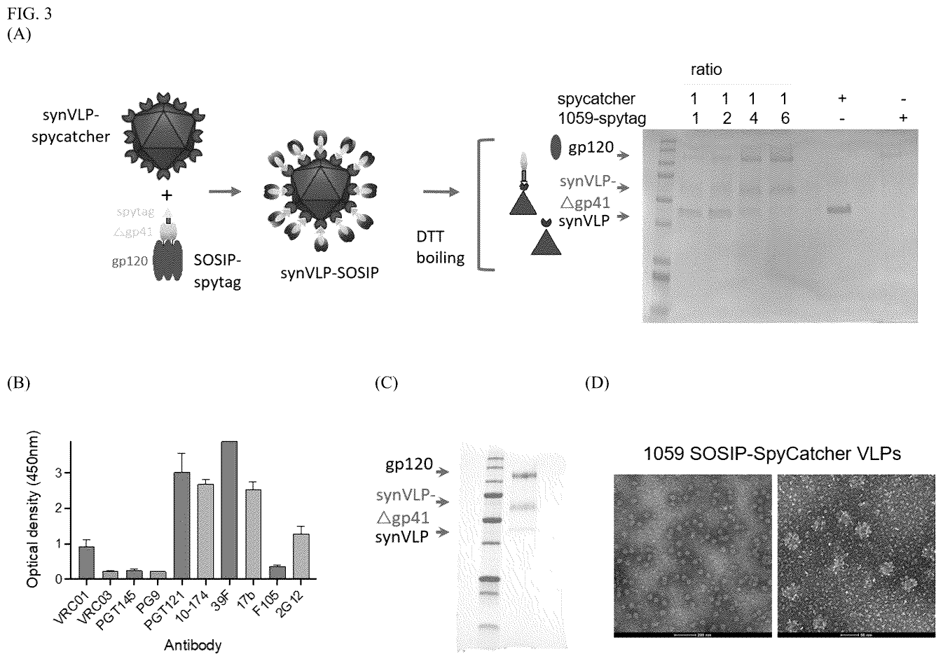

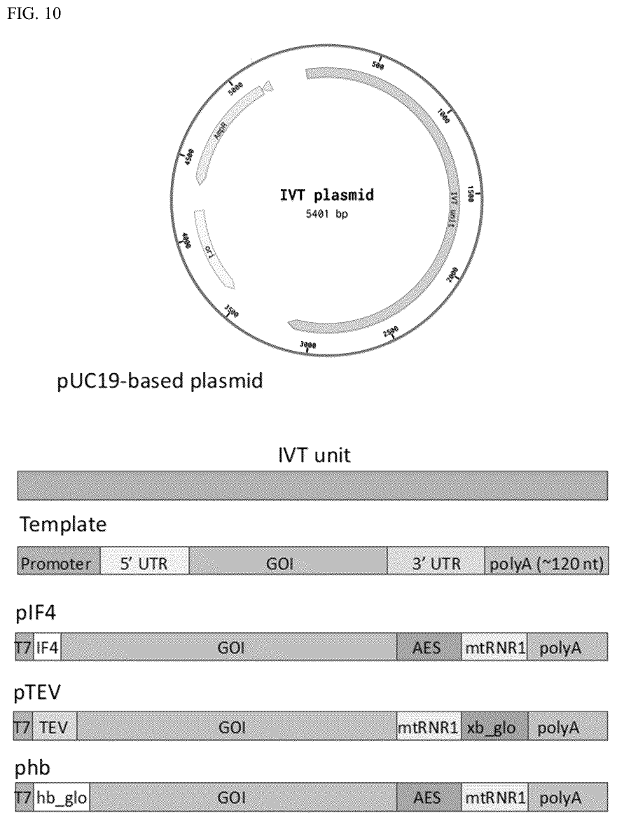

VLPs displaying HIV-1 Env immunogens were prepared using two platforms: mRNA-LNPs and synthetic icosahedral nanocages (synVLP). A new vector (FIG. 10) was constructed for in vitro transcription of mRNA for the expression of HIV-1_{AD8} Envs derived from an HIV-1 strain (AD8) that has been extensively studied in a non-human primate model of HIV-1 infection. The level of Env expression in 293T cells was confirmed and the entry-compatibility of mRNA-mediated Env expression was validated by producing PVs using the mRNA as a source for the Envs (FIG. 2). HIV-1_{AD8} PVs were highly infectious and, notably, removal of the cytoplasmic tail (ΔCT) of HIV-1_{AD8} Envs significantly increased viral infectivity, indicating that the HIV-1_{AD8} ΔCT Envs were maintaining entry-compatible conformation on the pseudovirus surface. mRNA-LNPs that mediate expression of HIV-1_{AD8} ΔCT Envs successfully supported the production of infectious PVs with a dose dependent infectivity within a range of 1 ng to 10 ng p24 levels (FIG. 2A). In parallel, a multi-valent display of 1059-SOSIP was built on synthetic nanocages using an available published system that is based on the Spytag-Spy Catcher technology (FIG. 3; Rahikainen et al., 2021, Angewandte Chemie International Edition 60:321-330; Bruun et al. 2018, ACS Nano 12:8855-8866).

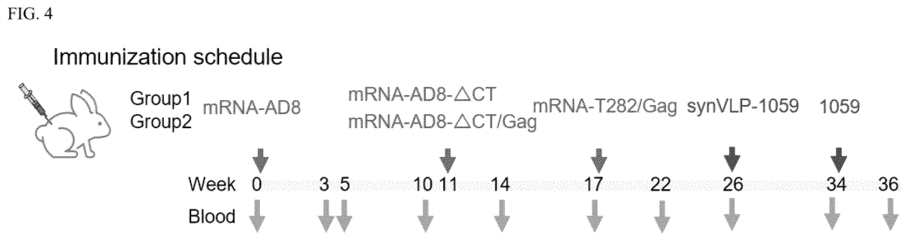

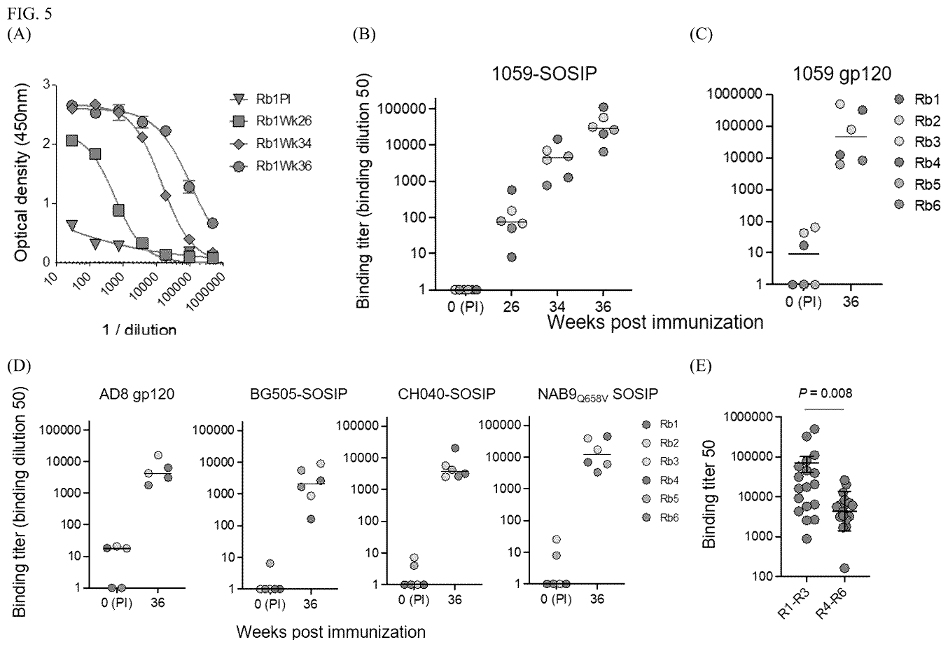

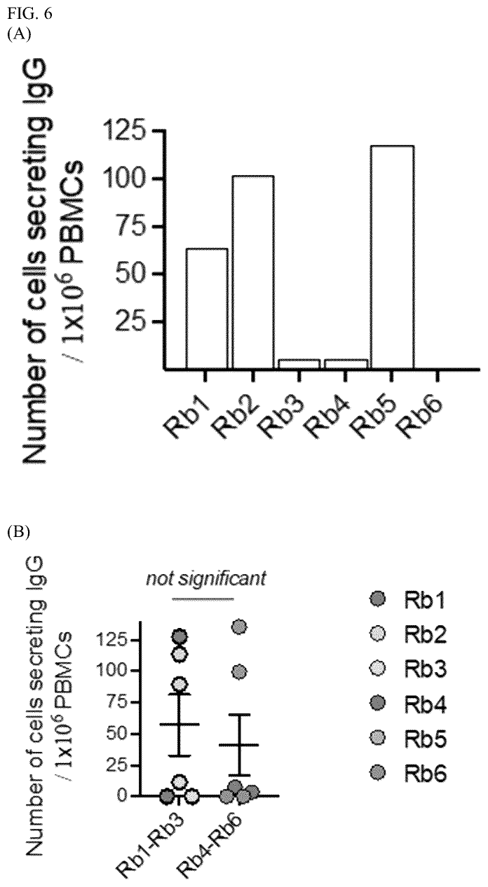

Next, six rabbits were immunized with a combination of different vectors that delivered HIV-1 Env immunogens at different conformations. All rabbits were primed with mRNA-LNPs, boosted twice with different mRNA-LNPs, and then sequentially boosted with syn VLP-1059 SOSIP followed by a boost of soluble 1059-SOSIP (FIG. 4). Rabbits were divided into two groups with only one difference between the groups: Group 1 was boosted (first boost) with mRNA-LNPs for expression of HIV-1_{AD8} ΔCT Envs whereas Group 2 was boosted (first boost) with mRNA-LNPs for co-expression of HIV-1_{AD8} ΔCT Envs and HIV-1 Gag in target cells. mRNA-mediated co-expressing HIV-1_{AD8} ΔCT Envs and HIV-1 Gag in the same cell produces viral-like particles displaying the ΔCT HIV-1_{AD8} Envs on their surfaces in vitro (FIG. 2C). Blood was collected at specified intervals (FIG. 4) and analyzed the serum for the antibody response. All rabbits developed high affinity antibodies over the time of immunization, with high titer (>1:10,000) against the 1059-SOSIP at week 36 (FIG. 5A, FIG. 5B). The median titer of all sera increased by 58.8-fold after the syn VLP boost (median ID₅₀ 1:74 vs 1:4,350) and further by 6.6-fold after the soluble 1059-SOSIP boost (median ID₅₀ 1:4,350 vs 1:28,738). Serum antibodies from all rabbits at week 36 bound to 1059-SOSIP at comparable levels to their binding to 1059 gp120 (FIG. 5B, FIG. 5C), suggesting that most of the antibody response was directed against gp120. Further, these data suggest that antibodies did not target a “glycan hole” in the SOSIP trimer that is a dominant immunogen observed for other SOSIP immunizations but absent in the gp120 monomer. Binding antibody response was broad as judged by sera binding to the heterologous BG505, CH040, and NAB9_{Q658V} SOSIPs and to the homologous AD8 gp120, although the titer in most of these cases was significantly lower compared with the titer for binding to the 1059 Envs (SOSIP or gp120). Notably, a significant difference between the two groups of rabbits was identified: sera from group 1 exhibited significantly stronger Env binding (higher binding titer 50) compared to sera from group 2 (FIG. 5E). A higher average number of antibody secreting cells were detected in group 1 compared to group 2.

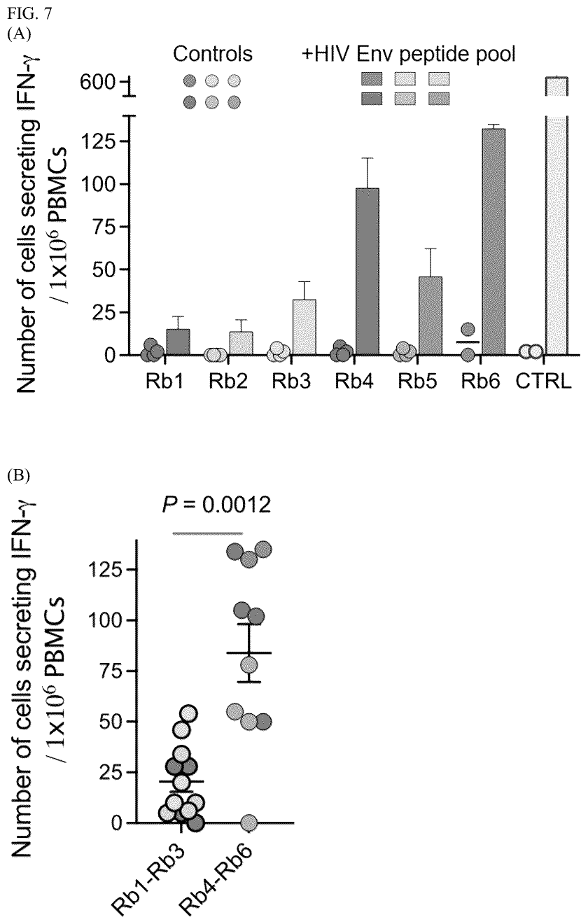

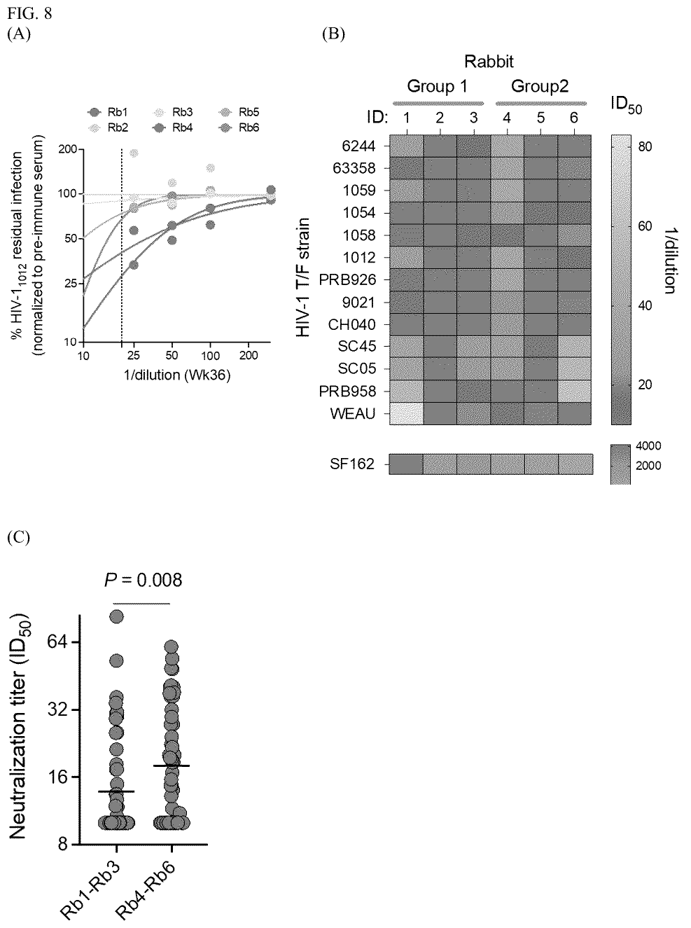

The frequency of PBMCs that were specific for HIV-1 Envs was evaluated by measuring the number of cells that were activated by a pool of HIV-1 Env peptides (ELISpot). PBMCs from all three rabbits of group 2 contained a higher number of HIV-1 Env-specific cells than PBMCs from any of the rabbits in group 1, and the difference between the two groups was statistically significant (FIG. 7A, FIG. 7B). Moreover, two patterns of reactivity were identified: group 1 showed high binding to soluble Envs but low cellular anti-Env response whereas group 2 exhibited the reverse phenotype with relatively lower binding to soluble Envs but high cellular anti-Env response. To assess the contribution of the cellular response to the development of a neutralization response to HIV-1, the ID₅₀ of all rabbit sera were measured against viruses pseudotyped with different Envs. Sera from all six rabbits efficiently neutralized the easy-to-neutralize HIV-1_{SF162} pseudoviruses at high titer (ID₅₀>1:2000 at week 36; FIG. 8B). None of the sera neutralized any of the 13 T/F entry very efficiently but sera from a subset of rabbits exhibited low titer but broad neutralization activity. In particular, low-titer serum of rabbit 4 (Rb4) neutralized 11 out of 13 T/F strains with a geometric mean of 1:27.2 over all 13 T/F Envs. There was no correlation between sera binding and efficient neutralization; sera of Rb1 exhibited the strongest binding (lower dilution) and sera of Rb4 and Rb6 exhibited the lowest binding whereas all three sera showed some low-titer neutralization activity. In contrast, Sera of Rb2, Rb3, and R5 exhibited intermediate binding among the six sera tested but showed no, or almost no, neutralization effects. Importantly, a strong association between the frequency of specific anti-Env PBMCs and viral neutralization activity was identified. Global analysis of sensitivity of all 13 T/F strains to rabbit sera from each group showed that inclusion of HIV-1 Gag in Group 2 (Rb4-Rb6) was beneficial and resulted in broader neutralization and statistically significant higher neutralization titer (FIG. 8B, FIG. 8C; P=0.008).

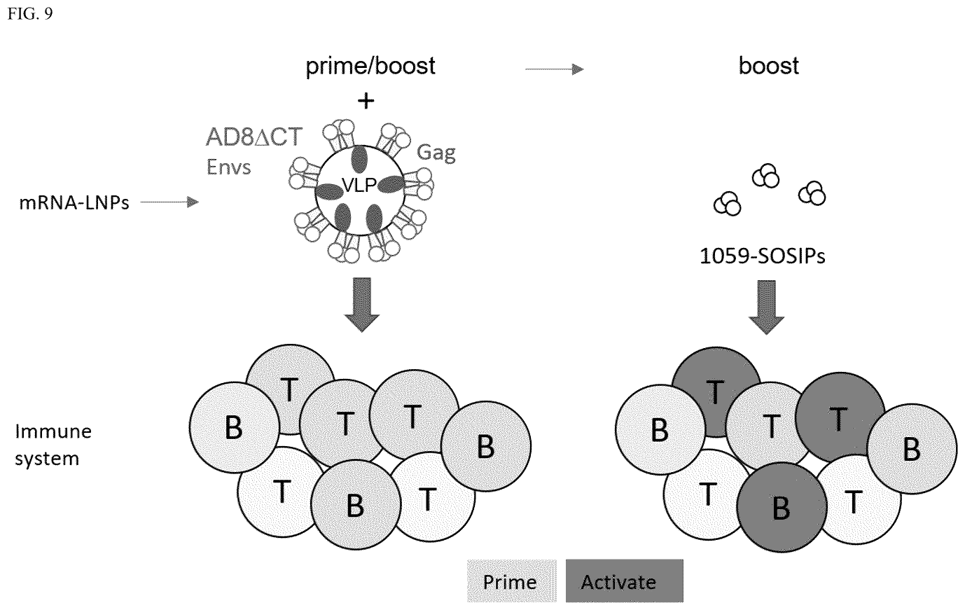

Development of an effective HIV-1 vaccine is extremely challenging but significant progress has been made in recent years due to incremental efforts and guiding insights. This disclosure shows that a sequential immunization scheme with conformationally diverse Envs results in low titer but broad neutralization activity in a subset of rabbits. One out of two rabbits in the preliminary study and three out of the six rabbits in the subsequent study developed low-titer antiviral antibody response. Moreover, this disclosure describes identification of a potential trigger for eliciting a neutralization response that is associated with high cellular response but not with high levels of sera binding to soluble Envs. mRNA-mediated co-expression of HIV-1_{AD8} ΔCT Envs and HIV-1 Gag induced a weak but broad neutralization response. It is unlikely that mRNA-mediated HIV-1 Gag expression by itself could be responsible for this effect as all rabbits were immunized (second boost; third immunization) with mRNA for co-expression of HIV-1 Gag with a full-length Envs from a strain other than HIV-1_{AD8}. Rather, the exact combination of mRNA-based, and co expression of HIV-1_{AD8} ΔCT Envs and HIV-1 Gag was required to enhance the neutralization response. HIV-1_{AD8} Envs were isolated from a macrophage/T cell tropic strain (infects both macrophages and T cells), are very stable and are expressed at high levels on the cell surface. Typically, ΔCT Env variants are expressed at 10-fold higher levels that full-length WT Envs and, specifically, HIV-1_{AD8} ΔCT Envs on pseudoviruses resulted in viruses that were significantly more infectious than HIV-1_{AD8} WT pseudoviruses (FIG. 2A). Thus, high levels of entry compatible Envs displayed on VLPs presented the Env immunogen to the immune system and triggered the development of an enhanced neutralizing response, which was associated with high frequency of anti-Envs PBMCs (FIG. 9).

Overall, a distinct pattern of humoral immune response was evident in each of the two groups of rabbits: the antibody response in group 1 was channeled toward high binding antibodies to soluble Envs whereas the response in group 2 efficiently activated a cellular anti-Env response and elicited neutralizing antibodies without the requirement to elicit high binding to soluble Envs. This approach may be further used to manipulate the type of antibody elicited by vaccination.

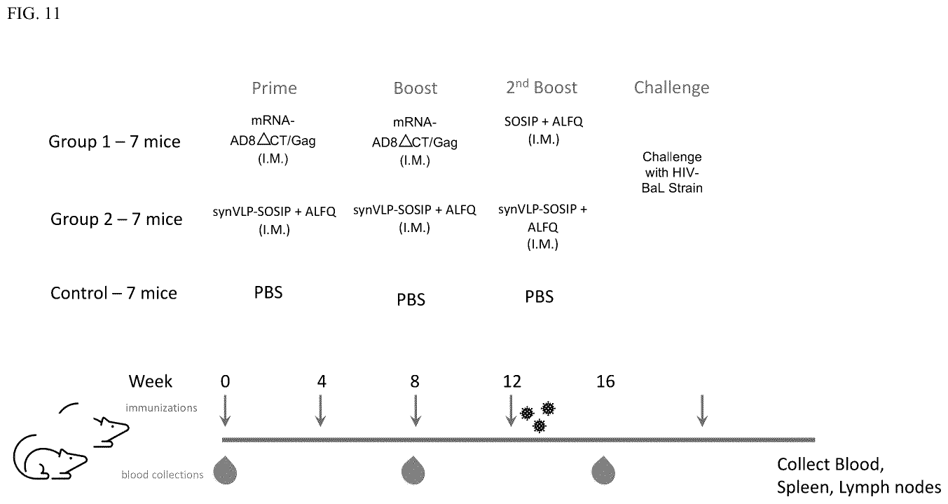

In a subsequent study, 21 DRAGA mice were divided into three groups. Each group was immunized with a different combination of vectors that presented HIV-1 Env immunogens to the immune system (FIG. 11). The mice of Group 1 were primed with an intramuscular injection of mRNA-LNPs for co-expression of HIV-1_{AD8} ΔCT Envs and HIV-1 Gag in target cells, boosted a first time at week 4 with mRNA-LNPs for co-expression of HIV-1_{AD8} ΔCT Envs and HIV-1 Gag, and boosted a second time at week 8 with soluble SOSIP and ALFQ. The mice of Group 2 were primed with syn VLP-SOSIP and ALFQ, boosted a first time at week 4 with synVLP-SOSIP and ALFQ, and boosted a second time at week 8 with syn VLP-SOSIP and ALFQ. The mice of Group 3, the control, were primed with PBS and boosted with PBS at weeks 4 and 8. A subset of mice were euthanized due to protocol health end point. Surviving members of all three groups were challenged with HIV-BaL Strain at week 12. Blood was collected at weeks 0, 8, and 16, and the serum was analyzed for antibody response. Subsequently, spleen and lymph nodes were collected.

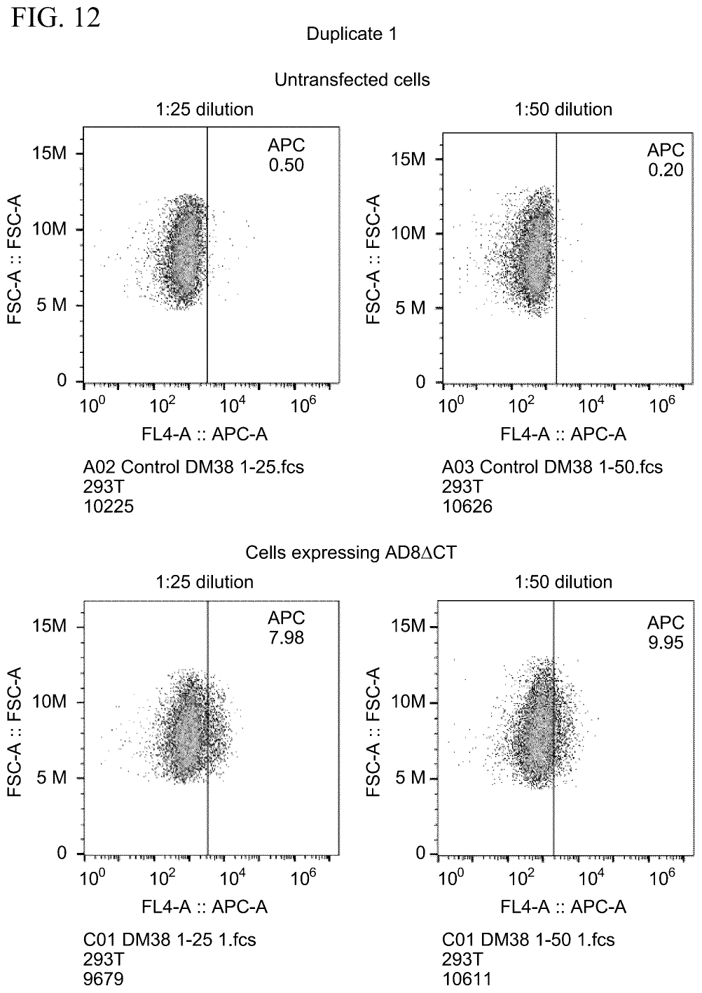

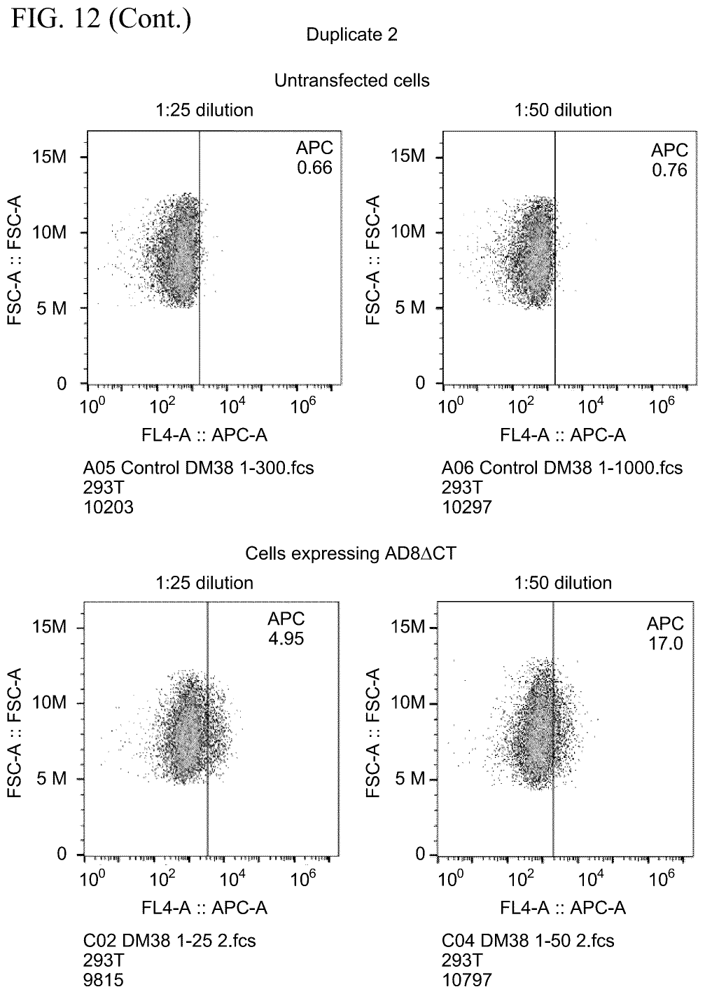

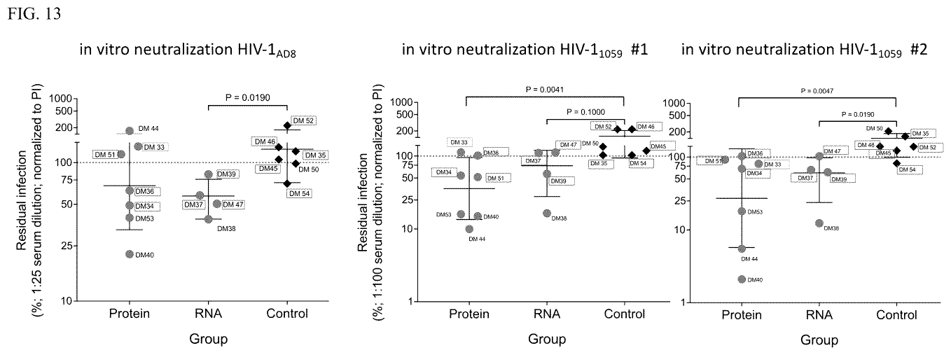

Neutralization activity of all mice sera was measured against viruses pseudotyped with HIV-1_{AD8} Envs and HIV-1₁₀₅₉ Envs (FIG. 13). FIG. 12 is a representative flow cytometry plot that reflects the binding of humanized DRAGA mouse #38 serum antibodies after immunization to HIV-1 envelope glycoproteins expressed on 293T cells. Humanized DRAGA mouse #38 belonged to Group I, meaning it was vaccinated with mRNA-LNPs for co-expression of HIV-1_{AD8} ΔCT Envs and HIV-1 Gag. Serum from humanized DRAGA mouse #38 showed the most promising neutralization activity, which serves as a proof of principle for VLP-based vaccines.

In one aspect, this disclosure provides novel methods for developing VLP-based vaccines against viral targets. VLP display of truncated envelope proteins were highly infectious, more so that VLP displaying full length envelope proteins, indicating that ΔCT Envs maintain entry-compatible conformation on the pseudovirus surface. While exemplified in the context of developing an HIV vaccine, the display of truncated envelope proteins by VLPs provides a broad platform for the development of vaccines against any enveloped virus by VLP display of truncated envelope proteins.

Generally, the method includes transfecting a cell with a combination of mRNAs to produce VLPs that display a truncated viral envelope protein on the surface of the VLP. The cell may be transfected in vitro or in vivo. Further, the combination of mRNAs may be introduced into the cell together or separately. When they are introduced separately, each mRNA may be introduced using a transfection method independent of the transfection method used to introduce any other mRNA into the cell. Generally, the cell is transfected with a polynucleotide that supports expression of a VLP subunit protein and a truncated form of a viral envelope protein natively expressed by the target virus. The truncated form (ΔCT) has a deletion of at least a portion of the cytoplasmic tail of the envelope protein. Once the cell is transfected, the cell is incubated under conditions effective to allow the cell to express both proteins. Typically, the VLP subunits are capable of self-assembly into the VLP displaying the ΔCT envelope protein on the surface of the VLP.

A variety of methods are known in the art and suitable for transfecting a cell by introducing a polynucleotide into a cell. Exemplary techniques include, but are not limited to, electroporation, calcium phosphate mediated transfer, nucleofection, sonoporation, heat shock, magnetofection, liposome mediated transfer, microinjection, microprojectile mediated transfer (nanoparticles), cationic polymer mediated transfer (DEAE-dextran, polyethylenimine, polyethylene glycol (PEG) and the like) or cell fusion. Other methods of transfection include proprietary transfection reagents such as LIPOFECTAMINE (Thermo Fisher Scientific, Inc., Waltham, MA), HILYMAX (Dojindo Molecular Technologies, Inc., Rockville, MD), FUGENE (Promega Corp., Madison, WI), JETPEI (Polyplus Transfection, Illkirch, France), EFFECTENE (Qiagen, Hilden, Germany) and DreamFect (OZ Biosciences, Inc., San Diego, CA).

The polynucleotide constructs described herein may be introduced into a host cell, thus allowing the host cell to express the protein encoded by the polynucleotide. A variety of host cells are known in the art and suitable for protein expression. Exemplary cells used for transfection and protein expression include, but are not limited to, a bacterial cell, a eukaryotic cell, a yeast cell, an insect cell, or a plant cell such as, for example, E. coli, Bacillus, Streptomyces, Pichia pastoris, Salmonella typhimurium, Drosophila S2, Spodoptera SJ9, CHO, COS (e.g., COS-7), 3T3-F442A, HeLa, HUVEC, HUAEC, NIH 3T3, Jurkat, 293, 293H, or 293F.

Given any amino acid sequence, a person of ordinary skill in the art can determine the full scope of polynucleotides that encode that amino acid sequence using conventional, routine methods.

As used herein, the term “polynucleotide” refers to any sequence of two or more nucleotides such as deoxyribonucleic acid (DNA) or ribonucleic acid (RNA). Polynucleotides include, but are not limited to, genomic DNA, cDNA, mRNA, iRNA, miRNA, tRNA, ncRNA, rRNA, and recombinantly produced and chemically synthesized molecules such as aptamers, plasmids, anti-sense DNA strands, shRNA, ribozymes, nucleic acids conjugates, and oligonucleotides. A polynucleotide may be single-stranded, double-stranded, linear, or covalently circularly closed molecule. A polynucleotide can be isolated. The term “isolated polynucleotide” means that the polynucleotide (i) was amplified in vitro, for example via polymerase chain reaction (PCR), (ii) was produced recombinantly by cloning, (iii) was purified, for example, by cleavage and separation by gel electrophoresis, (iv) was synthesized, for example, by chemical synthesis, or (vi) extracted from a sample. A polynucleotide might be introduced—i.e., transfected—into cells. When RNA is used to transfect cells, the RNA may be modified by stabilizing modifications, capping, or polyadenylation.

Generally, a polynucleotide can be extracted, isolated, amplified, or analyzed by a variety of techniques such as those described by Green and Sambrook, Molecular Cloning: A Laboratory Manual (Fourth Edition), Cold Spring Harbor Laboratory Press, Woodbury, NY 2,028 pages (2012); or as described in U.S. Pat. Nos. 7,957,913; 7,776,616; 5,234,809; and 9,012,208.

In another aspect, this disclosure describes compositions for treating a subject having, or at risk of having an infection by an enveloped virus. In one or more embodiments, the composition includes a virus-like particle (VLP) and a truncated form (ΔCT) of a viral envelope protein displayed on the VLP, in which the truncated form of the viral protein has a deletion of at least a portion of the cytoplasmic tail region of the envelope protein.

In one or more alternative embodiments, the composition includes a first polynucleotide that encodes a VLP subunit protein, a second polynucleotide that encodes a truncated form (ΔCT) of a viral envelope protein, and a delivery vehicle attached to or encapsulating the first polynucleotide and the second polynucleotide. In one or more of these embodiments, the delivery vehicle can be a lipid nanoparticle.

As used herein, “treat” or variations thereof refer to reducing, limiting progression, ameliorating, or resolving, to any extent, the symptoms or signs related to a condition. As used herein, “symptom” refers to any subjective evidence of disease or of a patient's condition; “sign” or “clinical sign” refers to an objective physical finding relating to a particular condition capable of being found by one other than the patient. A “treatment” may be therapeutic or prophylactic. “Therapeutic” and variations thereof refer to a treatment that ameliorates one or more existing symptoms or clinical signs associated with a condition. “Prophylactic” and variations thereof refer to a treatment that limits, to any extent, the development and/or appearance of a symptom or clinical sign of a condition. Generally, a “therapeutic” treatment is initiated after the condition manifests in a subject, while “prophylactic” treatment is initiated before a condition manifests in a subject.

Treatment that is prophylactic—e.g., initiated before a subject manifests a symptom or clinical sign of the condition such as, for example, while an infection remains subclinical—is referred to herein as treatment of a subject that is “at risk” of having the condition. As used herein, the term “at risk” refers to a subject that may or may not actually possess the described risk. Thus, for example, a subject “at risk” of infectious condition is a subject present in an area where other individuals have been identified as having the infectious condition and/or is likely to be exposed to the infectious agent (e.g., a virus) even if the subject has not yet manifested any detectable indication of infection by the infectious agent and regardless of whether the subject may harbor a subclinical amount of the infectious agent. As another example, a subject “at risk” of an infectious condition is a subject possessing one or more risk factors associated with increased risk of infection and/or serious complications upon infection by the infectious agent such as, for example, genetic predisposition, ancestry, age, sex, geographical location, lifestyle, or medical history. Treatment may also be continued after symptoms have resolved, for example to prevent or delay their recurrence.

Accordingly, a composition can be administered before, during, or after the subject first exhibits a symptom or clinical sign of the condition or before, during, or after the subject first comes in contact with the infectious agent. Treatment initiated before the subject first exhibits a symptom or clinical sign associated with the condition may result in decreasing the likelihood that the subject experiences clinical evidence of the condition compared to a subject to which the composition is not administered, decreasing the severity of symptoms and/or clinical signs of the condition compared to a subject to which the composition is not administered, and/or completely resolving the condition. Treatment initiated after the subject first exhibits a symptom or clinical sign associated with the condition may result in decreasing the severity of symptoms and/or clinical signs of the condition compared to a subject to which the composition is not administered, and/or completely resolving the condition.

Thus, the method includes administering an effective amount of the composition to a subject having, or at risk of having, a viral infection. In this aspect, an “effective amount” is an amount effective to reduce, limit progression, ameliorate, or resolve, to any extent, a symptom or clinical sign related to the viral infection.

The subject can be a human or a non-human animal such as, for example, a livestock animal, a laboratory animal, or a companion animal. Exemplary non-human animal subjects include, but are not limited to, animals that are hominid (including, for example chimpanzees, gorillas, or orangutans), bovine (including, for instance, cattle), caprine (including, for instance, goats), ovine (including, for instance, sheep), porcine (including, for instance, swine), equine (including, for instance, horses), members of the family Cervidae (including, for instance, deer, elk, moose, caribou, or reindeer), members of the family Bison (including, for instance, bison), feline (including, for example, domesticated cats, tigers, lions, etc.), canine (including, for example, domesticated dogs, wolves, etc.), avian (including, for example, turkeys, chickens, ducks, geese, etc.), a rodent (including, for example, mice, rats, etc.), a member of the family Leporidae (including, for example, rabbits or hares), members of the family Mustelidae (including, for example ferrets), or member of the order Chiroptera (including, for example, bats).

The compositions described herein, whether including VLPs or polynucleotides that encode VLP components (collectively referred to as “active agents”), may be formulated with a pharmaceutically acceptable carrier. As used herein, “carrier” includes any solvent, dispersion medium, vehicle, coating, diluent, antibacterial, and/or antifungal agent, isotonic agent, absorption delaying agent, buffer, carrier solution, suspension, colloid, and the like. The use of such media and/or agents for pharmaceutical active substances is well known in the art. Except insofar as any conventional media or agent is incompatible with the active ingredient (e.g., the VLPs or polynucleotides encoding VLP components), its use in the therapeutic compositions is contemplated. Supplementary active ingredients also can be incorporated into the compositions. As used herein, “pharmaceutically acceptable” refers to a material that is not biologically or otherwise undesirable, i.e., the material may be administered to an individual along with the active agent without causing any undesirable biological effects or interacting in a deleterious manner with any of the other components of the pharmaceutical composition in which it is contained.

The active agent or active agents may therefore be formulated into a pharmaceutical composition. The pharmaceutical composition may be formulated in a variety of forms adapted to a preferred route of administration. Thus, a composition can be administered via known routes including, for example, oral, parenteral (e.g., intradermal, transcutaneous, subcutaneous, intramuscular, intravenous, intraperitoneal, etc.), or topical (e.g., intranasal, intrapulmonary, intramammary, intravaginal, intrauterine, intradermal, transcutaneous, rectally, etc.). A pharmaceutical composition can be administered to a mucosal surface, such as by administration to, for example, the nasal or respiratory mucosa (e.g., by spray or aerosol). A composition also can be administered via a sustained or delayed release.

Thus, the active agent or active agents may be provided in any suitable form including but not limited to a solution, a suspension, an emulsion, a spray, an aerosol, or any form of mixture. The composition may be delivered in formulation with any pharmaceutically acceptable excipient, carrier, or vehicle. For example, the formulation may be delivered in a conventional topical dosage form such as, for example, a cream, an ointment, an aerosol formulation, a non-aerosol spray, a gel, a lotion, and the like. The formulation may further include one or more additives including, but not limited to, an adjuvant, a skin penetration enhancer, a colorant, a fragrance, a flavoring, a moisturizer, a thickener, and the like.

A formulation may be conveniently presented in unit dosage form and may be prepared by methods well known in the art of pharmacy. Methods of preparing a composition with a pharmaceutically acceptable carrier include the step of bringing the active agent or active agents into association with a carrier that constitutes one or more accessory ingredients. In general, a formulation may be prepared by uniformly and/or intimately bringing the active compound into association with a liquid carrier, a finely divided solid carrier, or both, and then, if necessary, shaping the product into the desired formulations.

The amount of VLPs or polynucleotides encoding VLP components administered can vary depending on various factors including, but not limited to, the specific active agent or active agents being administered, the weight, physical condition, and/or age of the subject, and/or the route of administration. Thus, the absolute amount of active agent or active agents included in a given unit dosage form can vary widely, and depends upon factors such as the species, age, weight, and physical condition of the subject, and/or the method of administration. Accordingly, it is not practical to set forth generally the amount that constitutes an amount of the active agent or active agents effective for all possible applications. Those of ordinary skill in the art, however, can readily determine the appropriate amount with due consideration of such factors.

A single dose may be administered all at once, continuously for a prescribed period of time, or in multiple discrete administrations. When multiple administrations are used, the amount of each administration may be the same or different. When multiple administrations are used to deliver a single dose, the interval between administrations may be the same or different.

In one or more embodiments, the active agent may be administered, for example, from a single dose to multiple doses over a prescribe period of time. When a course of treatment involves administering multiple doses within a certain period of time, the amount of each dose may be the same or different. For example, a course of treatment can include an initial loading dose, followed by a maintenance dose or a booster dose that is lower than the loading dose. Also, when multiple doses are used within a certain period, the interval between doses may be the same or be different.

### EXAMPLES

The present invention is illustrated by the following examples. It is to be understood that the particular examples, materials, amounts, and procedures are to be interpreted broadly in accordance with the scope and spirit of the invention as set forth herein.

### Plasmid Construction

All plasmids for soluble SOSIP Env expression were codon-optimized and included the tissue plasminogen activator (TPA) signal peptide instead of the natural signal peptide. In vitro transcription (IVT) cassettes for mRNA synthesis were designed in-house and included bacteriophage T7 RNA polymerase promoter, stable untranslated regions (UTRs) based on previously published reports (Orlandini von Niessen et al., 2019, Mol Ther 27:824-836; Gallie et al., 1995, Gene 165:233-238; Tusup et al., 2019, Chimia (Aarau) 73:391-394), gene-of-interest, and a 125-base polyA tail (FIG. 10). Designed DNA was synthesized and cloned into a pUC19 plasmid by Gene Universal (Newark, DE). All plasmids were fully sequenced to verify correct sequence of the IVT cassettes. IVT plasmid for mRNA-mediated expression of HIV-1_{AD8} ΔCT Envs was generated by digesting the parental HIV-1_{AD8} WT env plasmid with BsiWI restriction enzyme (2 sites) to remove the CT DNA-encoding fragment and self-ligating the digested vector. The resulting vector contained only 120-base polyA tail due to bacteria-mediated deletion during generation of this variant.

### Protein Expression and Purification

293F cells were co-transfected with a SOSIP-expressing plasmid and a human furin-expressing plasmid at ratio of 4:1 using Turbo293 transfection reagent (Speed Biosystems LLC, Gaithersburg, MD). Transfected cells were grown on a shaker in a tissue culture incubator at 37° C., 8% CO₂ for 3-5 days and culture supernatants were then harvested, clarified by centrifugation at 4000×g for 20 minutes and filtered through 0.2 μm filter (VWR, Avantor, Inc., Radnor, PA). Supernatant containing SOSIP glycoproteins was loaded on Galanthus nivalis lectin (GNL) column (Vector Laboratories, Inc., Newark, CA) at 4-8° C., the column was washed with 500 mM NaCl in phosphate buffered saline pH 8 and proteins were eluted with 1M Methyl-α-D-mannopyranoside/PBS solution, filtered through 0.2 μm filter and concentrated using a centrifugal concentrator (VIVASPIN 6, Sartorius A G, Göttingen, Germany; 30 kDa). Purified SOSIP glycoproteins were then separated on a size exclusion chromatography column (HILOAD 16/600 SUPERDEX, Cytiva, Marlborough, MA; 200 pg) and SOSIP trimer fractions were pooled, concentrated, and stored in aliquots at −80° C. until use.

### ELISA

Enzyme-linked immunosorbent assay (ELISA) was used to analyze rabbit sera binding to SOSIP trimers. GNL was immobilized in a 96-well plate (Greiner Bio-One International GmbH, Kremsmünster, Austria) by adding 0.25 μg of GNL in 100 μl PBS in each well and incubating the plates overnight at room temperature (RT). Next, the wells were washed three times with PBS containing 0.2% Tween-20 (wash solution) using in-house vacuum system and blocked with PBS containing 3% bovine serum albumin (blocking solution) for two hours at RT. The wells were then washed three times, 0.2-0.25 μg of purified SOSIP trimers in blocking solution were added to test wells and the plate was incubated for one-to-two hour(s) at RT. Wells were washed six times and different dilutions of rabbit sera in blocking solution were added. After a 90-minute incubation, wells were washed six times and 1:50,000 dilution of horseradish peroxidase (HRP)-conjugated donkey anti-rabbit IgG (FC specific; Jackson ImmunoResearch Laboratories, Inc., West Grove, PA) was added in blocking solution to each well and the plate was incubated for one hour at RT. Wells were then washed six times and 100 μl of TMB solution (1 ml of 1 mg/ml 3,3,5,5-tetramethylbenzidine (Sigma) in DMSO, 9 ml of 0.1 M sodium acetate, pH 5.0, and 2 μl of fresh 30% hydrogen peroxide) was added to each well. After ˜18-minute incubation, the HRP reaction was stopped by adding 50 μl of 0.5 M H₂SO₄ and optical density at 450 nm was measured using a spectrophotometer.

In Vitro Transcription, mRNA Purification, and mRNA-LNP Preparation

mRNA was simultaneously in vitro transcribed and capped using the T7-FLASHSCRIBE transcription kit (CellScript, LLC, Madison, WI) and CLEANCAP reagent AG (TriLink BioTechnologies, San Diego, CA) according to the manufacturers' instructions. In some cases, modified pseudouridine (N1-methyl-pseudouridine-5′-triphosphate) were used instead of uridine-5′-triphosphate to stabilize the mRNA molecules. Transcribed mRNA was purified using MEGACLEAR transcription clean-up kit (Thermo Fisher Scientific, Inc., Waltham, MA) according to the manufacturer's instructions and impurities were further removed on cellulose columns as previously described (Baiersdörfer et al., 2019, Mol Ther Nucleic Acids 15:26-35). mRNA concentration was measured by optical density at 260 nm and the mRNA preparation was frozen at −80° C. until it was encapsulated by LNPs (Acuitas Therapeutics, Inc., Vancouver, British Columbia, Canada) and stored in single-use tubes at −80° C.

### Animal Care

Experiments involving New Zealand White rabbit were carried out according to NIH guidelines for the housing and care of laboratory animals. Protocols were reviewed and approved by the Institutional Animal Care and Use Committee (IACUC) of the University of Minnesota.

### Rabbit Immunizations

Rabbits were intramuscularly immunized in a single site in the quadriceps muscle with either mRNA-LNPs (35 μg/rabbit), synVLP-1059 SOSIP (21.5 μg/rabbit) or soluble SOSIP trimers (50 μg/rabbit). Protein immunizations were administered after mixing the protein solution (PBS) with ADDAVAX adjuvant (InvivoGen, San Diego, CA) at 1:1 volume ratio. Due to technical error, during the first immunization with mRNA-LNPs, rabbit 6 was initially immunized with only 11 μg and the additional 24 μg were administered one week later.

### Production of Single-Round Pseudoviruses

Pseudoviruses were produced as we previously described (Herschhorn et al., 2016, mBio 7:1-12; Herschhorn et al., 2017, Nature Communications 8:1049; Harris et al., 2020, Cell Reports 31:107749; Herschhorn et al., 2010, J Immunol 185:7623-7632). Briefly, a packaging plasmid (psPAX2), a reporter plasmid (pHIVec2.luc), and an Env-expressing plasmid were co-transfected into 293T cells using EFFECTENE transfection reagent (Qiagen, Hilden, Germany). After a 48-hour incubation, the cell supernatant was collected and centrifuged for five minutes at 600-900×g at 4° C. The amount of p24 in the supernatant was measured using the HIV-1 p24 antigen capture assay (Cat #5421, Advanced BioScience Laboratories, Inc., Rockville, MD) and the virus-containing supernatant was frozen in single-use aliquots at −80° C. In some cases, the Env-expressing plasmid was replaced by Env-expressing mRNA that was transfected using Trans-IT (Mirus Bio LLC, Madison, WI) or by the direct addition of mRNA-LNPs, both 24 hours post transfection of the packaging and reporter DNA plasmids.

### Viral Infection Assay

A single-round infection assay was performed in 96-well white plates (Greiner Bio-One International GmbH, Kremsmünster, Austria) using TZM-bl target cells (NIH AIDS Reagent Program). 30 μl of diluted serum was added to each well followed by addition of 30 μl of specified pseudoviruses and the plate was incubated for one hour at 37° C. in a tissue culture incubator with 5% CO₂. Then, approximately 7,000 TZM-bl cells in 30 μl DMEM were added to each well, the plate was incubated for 48 hours, cells lysed, and luciferase activity was measured by microplate luminometer (CENTRO XS³ LB 960, Berthold Technologies Gmbh & Co. KG, Bad Wildbad, Germany). Dose response curves were non-linearly fitted to the logistic equation using PRISM 9 (GraphPad Software, San Diego, CA), after importing the logistic equation to the analysis software, as previously described (Harris et al., 2020, Cell Reports 31:107749; Ratnapriya et al., 2020, STAR Protocols 1:100133; Herschhorn et al., 2011, PLoS ONE 6:10; Farzani et al., 2020, STAR Protocols 1:100209), except that doses were expressed as dilution instead of absolute concentration and reported parameters were half-maximal inhibitory dilution (ID₅₀).

### Peripheral Blood Mononuclear Cells (PBMCs) Isolation

Blood was collected from the ear vein of rabbits in 6 ml heparinized tubes and used immediately after collection. PBMCs were purified by density centrifugation (800×g for 30 minutes) on Lymphocyte separation medium 1077 (PromoCell GmbH, Heidelberg, Germany), isolated from the gradient interface, washed twice in Dulbecco's phosphate buffered saline (DPBS, Thermo Fischer Scientific, Inc., Waltham, MA), and resuspended in Roswell Park Memorial Institute (RPMI) 1640 medium (Thermo Fisher Scientific, Inc., Waltham, MA) supplemented with 10% heat-inactivated fetal bovine serum (FBS).

### ELISpot Assay

ELISpot assay was based on the ELISpot Flex: Rabbit IFN-γ (HRP) kit (Mabtech AB, Nacka Strand, Sweden). On day 1, a 96-well PVDF membrane white plate (Mabtech AB, Nacka Strand, Sweden) was pre-treated with 35% ethanol for one minute to activate the membrane and obtain maximal antibody binding capacity. The plates were then coated with 100 μl of 15 μg/ml unconjugated anti-rabbit IFN-γ mAb (MT327) in PBS and incubated overnight at 4° C. On day 2, the plate was washed five times with sterile PBS, and then blocked with RPMI 1640 medium at room temperature for at least 30 minutes. Freshly isolated PBMCs were added to the plate together with 2 μg/ml of the Peptide Pool, HIV-1 Subtype B (Consensus) Env Region (NIH-ARP Cat #12540). Medium-only samples were added as controls to assess background level of lymphokine secretion. PMA/ionomycin was added to positive control wells. The plate was incubated for 18 hours at 37° C. in 5% CO₂ tissue culture incubator. On day 3, the plate was washed with PBS and then incubated for two hours at room temperature with 0.1 μg/ml of biotinylated anti-rabbit IFN-γ mAb (MT318). The plate was then washed and incubated with 100 μl of Streptavidin-HRP (1:1000 dilution) for one hour at room temperature. ELISpot substrate (TMB; Mabtech AB, Nacka Strand, Sweden) was added until distinct spots appeared. The plate was dried at room-temperature overnight. On day 4, the number of spots in each well was measured using an IMMUNOSPOT analyzer (Cellular Technology Ltd., Shaker Heights, OH). Two different cell densities were used in duplicate (either 2×10⁵ cells/well or 5×10⁵ cells/well), normalized the results to frequency of cell in 1×10⁶ PBMCs and average the four replicates (two from each cell density).

### ELISpot IgG Assay

ELISpot IgG assay was based on the ELISpot Flex: Rabbit IgG (HRP) kit (Mabtech AB, Nacka Strand, Sweden). On day 1, a 96-well PVDF membrane white plate (Mabtech AB, Nacka Strand, Sweden) was pre-treated with 35% ethanol for one minute to activate the membrane and allow maximal antibody binding capacity. The plate was washed five times with sterile water and coated with 100 μl of 2.5 μg/ml unconjugated antigen (1059 SOSIP trimer) in PBS and incubated overnight at 4° C. On day 2, the plate was washed five times with sterile PBS, and then blocked with RPMI 1640 medium with 10% FBS at room temperature for at least 30 minutes. Freshly isolated PBMCs suspended in RPMI 1640 10% FBS were added to the plate and medium-only samples were added as controls to assess background level of IgG secretion. The plate was incubated for 20 hours at 37° C. in 5% CO₂ tissue culture incubator. On day 3, the plate was washed with PBS and then incubated for two hours at room temperature with 0.5 μg/ml of biotinylated detection mAb (MT536; Mabtech AB, Nacka Strand, Sweden) in PBS-0.5% FBS. The plate was then washed and incubated with 100 μl of Streptavidin-HRP (1:1000 dilution) for one hour at room temperature. ELISpot substrate (TMB) was added until spots were visible. The plate was dried at room-temperature overnight. On day 4, the number of spots in each well was measured using an IMMUNOSPOT analyzer (Cellular Technology Ltd., Shaker Heights, OH). 5×10⁵ cells/well were used in duplicate, background measurements of multiple wells (medium only) were subtracted, and the number of antibody secreting cells in 1×10⁶ PBMCs was calculated.

### Humanized DRAGA Mouse Immunization Scheme

21 humanized DRAGA mice were immunized with a combination of different vectors that delivered HIV-1 Env immunogens (FIG. 11). The mice were divided into three group of seven. The mice of Group 1 were primed with an intramuscular injection of mRNA-LNPs for co-expression of HIV-1_{AD8} ΔCT Envs and HIV-1 Gag in target cells, boosted a first time at week 4 with mRNA-LNPs for co-expression of HIV-1_{AD8} ΔCT Envs and HIV-1 Gag, and boosted a second time at week 8 with soluble SOSIP and ALFQ. The mice of Group 2 were primed with syn VLP-SOSIP and ALFQ, boosted a first time at week 4 with syn VLP-SOSIP and ALFQ, and boosted a second time at week 8 with syn VLP-SOSIP and ALFQ. The mice of Group 3, the control, were primed with PBS and boosted with PBS at weeks 4 and 8. Mice from each group were challenged with HIV-BaL Strain at week 12. Blood was collected at weeks 0, 8, and 16, and the serum was analyzed for antibody response. Subsequently, spleen and lymph nodes were collected.

### Humanized DRAGA Mouse Flow Cytometry and Neutralization Measurements

Neutralization activity of all mouse sera was measured against viruses pseudotyped with HIV-1_{AD8} Envs and HIV-1₁₀₅₉ Envs (FIG. 13). FIG. 12 is a representative flow cytometry plot that reflects the binding of post-immunization serum antibodies of humanized DRAGA mouse #38 (a Group I mouse) to HIV-1 envelope glycoproteins expressed on 293T cells.

### ILLUSTRATIVE EMBODIMENTS

Embodiment 1. A composition comprising:
a virus-like particle (VLP); and
a truncated form of a viral envelope protein displayed on the VLP, the truncated form comprising a deletion of at least a portion of a cytoplasmic tail region of the envelope protein.
   Embodiment 2. The composition of Embodiment 1, wherein:
the VLP comprises HIV-1 Gag; and
the truncated form of the viral envelope protein comprises HIV-1 envelope protein with at least a portion of the cytoplasmic tail deleted.
   Embodiment 3. The composition of Embodiment 1 or Embodiment 2, further comprising an adjuvant.
   Embodiment 4. A composition comprising:
a first polynucleotide encoding a VLP subunit protein;
a second polynucleotide encoding a viral envelope protein comprising a deletion of at least a portion of a cytoplasmic tail; and
a delivery vehicle attached to or encapsulating the first polynucleotide and the second polynucleotide together or separately.
   Embodiment 5. The composition of Embodiment 4, wherein the delivery vehicle comprises a lipid nanoparticle.
   Embodiment 6. A composition comprising:
a first polynucleotide attached to or encapsulated by a first delivery vehicle, the first polynucleotide encoding a VLP subunit protein;
a second polynucleotide attached to or encapsulated by a second delivery vehicle, the second polynucleotide encoding a viral envelope protein comprising a deletion of at least a portion of a cytoplasmic tail.
   Embodiment 7. The composition of Embodiment 6, wherein the first delivery vehicle, the second delivery vehicle, or both delivery vehicles comprises a lipid nanoparticle.
   Embodiment 8. A method of preparing a VLP-based vaccine, the method comprising:
transfecting a cell with a first polynucleotide encoding a VLP subunit protein;
transfecting the cell with a second polynucleotide encoding a truncated viral envelope protein comprising a deletion of at least a portion of a cytoplasmic tail;
allowing the cell to express the first polynucleotide to produce VLP subunits;
allowing the cell to express the second polynucleotide to produce the truncated viral envelope protein; and
allowing the VLP subunits and the truncated viral envelope protein to assemble into a VLP in which the truncated viral envelope protein is displayed on the surface of the VLP.
   Embodiment 9. The method of Embodiment 8, wherein the cell is transfected in vitro.
   Embodiment 10. The method of Embodiment 8, wherein the cell is transfected in vivo.
   Embodiment 11. The method of any one of Embodiments 8-10, wherein the cell is transfected with the first polynucleotide and the second polynucleotide simultaneously.
   Embodiment 12. The method of any one of Embodiments 8-10, wherein the cell is transfected with the first polynucleotide and the second polynucleotide separately.
   Embodiment 13. The method of any one of Embodiments 8-12, wherein transfecting the cell with the first polynucleotide or transfecting the cell with a second polynucleotide comprises using a lipid nanoparticle as a delivery vehicle.
   Embodiment 14. A method of treating a subject having, or at risk of having, an infection by an enveloped virus, the method comprising:
administering to the subject a composition in an amount effective to treat infection by the enveloped virus, the composition comprising:
a virus-like particle (VLP); and
a truncated form of a viral envelope protein displayed on the VLP, the truncated form comprising a deletion of at least a portion of a cytoplasmic tail region of the envelope protein.
   Embodiment 15. A method of treating a subject having, or at risk of having, an infection by an enveloped virus, the method comprising:
administering to the subject a composition in an amount effective to treat infection by the enveloped virus, the composition comprising:
a first polynucleotide encoding a VLP subunit protein;
a second polynucleotide encoding a viral envelope protein comprising a deletion of at least a portion of a cytoplasmic tail; and
a delivery vehicle attached to or encapsulating the first polynucleotide and the second polynucleotide.
   Embodiment 16. The method of Embodiment 15, wherein the delivery vehicle comprises a lipid nanoparticle.

In the preceding description and following claims, the term “and/or” means one or all of the listed elements or a combination of any two or more of the listed elements; the terms “comprises,” “comprising,” and variations thereof are to be construed as open ended—i.e., additional elements or steps are optional and may or may not be present; unless otherwise specified, “a,” “an,” “the,” and “at least one” are used interchangeably and mean one or more than one; and the recitations of numerical ranges by endpoints include all numbers subsumed within that range (e.g., 1 to 5 includes 1, 1.5, 2, 2.75, 3, 3.80, 4, 5, etc.).

In the preceding description, particular embodiments may be described in isolation for clarity. Reference throughout this specification to “one embodiment,” “an embodiment,” “certain embodiments,” “one or more embodiments,” or “some embodiments,” etc., means that a particular feature, configuration, composition, or characteristic described in connection with the embodiment is included in at least one embodiment of the disclosure. Thus, the appearances of such phrases in various places throughout this specification are not necessarily referring to the same embodiment of the disclosure. Furthermore, the particular features, configurations, compositions, or characteristics may be combined in any suitable manner in one or more embodiments. Thus, features described in the context of one embodiment may be combined with features described in the context of a different embodiment except where the features are necessarily mutually exclusive.

As used herein, the word “exemplary” means to serve as an illustrative example and should not be construed as preferred or advantageous over other embodiments.

The words “preferred” and “preferably” refer to embodiments of the invention that may afford certain benefits under certain circumstances. However, other embodiments may also be preferred under the same or other circumstances. Furthermore, the recitation of one or more preferred embodiments does not imply that other embodiments are not useful and is not intended to exclude other embodiments from the scope of the invention.

In several places throughout the above description, guidance is provided through lists of examples, which examples can be used in various combinations. In each instance, the recited list serves only as a representative group and should not be interpreted as an exclusive list.

For any method disclosed herein that includes discrete steps, the steps may be performed in any feasible order. And, as appropriate, any combination of two or more steps may be performed simultaneously.

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference in their entirety. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

Sequence Listing Free Text

SEQ ID NO: 1-HIV-1 envelope protein
MKVKGIRKNY QHLWKWGIML LGMLMICSAV ENLWVTVYYG VPVWKEATTT LFCASDAKAY
DTEVHNVWAT HACVPTDPNP QEVVLENVTE NFNMWKNNMV EQMHEDIISL WDQSLKPCVK
LTPLCVTLNC TDLRNVTNIN NSSEGMRGEI KNCSFNITTS IRDKVKKDYA LFYRLDVVPI
DNDNTSYRLI NCNTSTITQA CPKVSFEPIP IHYCTPAGFA ILKCKDKKFN GTGPCKNVST
VQCTHGIRPV VSTQLLINGS LAEEEVVIRS SNFTDNAKNI IVQLKESVEI NCTRPNNNTR
KSIHIGPGRA FYTTGDIIGD IRQAHCNISR TKWNNTLNQI ATKLKEQFGN NKTIVFNQSS
GGDPEIVMHS FNCGGEFFYC NSTQLFNSTW NFNGTWNLTQ SNGTEGNDTI TLPCRIKQII
NMWQEVGKAM YAPPIRGQIR CSSNITGLIL TRDGGNNHNN DTETFRPGGG DMRDNWRSEL
YKYKVVKIEP LGVAPTKAKR RVVQREKRAV GTIGAMFLGF LGAAGSTMGA ASITLTVQAR
LLLSGIVQQQ NNLLRAIEAQ QHLLQLTVWG IKQLQARVLA LERYLRDQQL LGIWGCSGKL
ICTTAVPWNA SWSNKTLDMI WNNMTWMEWE REIDNYTGLI YTLIEESQNQ QEKNEQELLE
LDKWASLWNW FDITNWLWYI KIFIMIVGGL IGLRIVFTVL SIVNRVRQGY SPLSFQTHLP
APRGPDRPEG IEEEGGDRDR DRSVRLVDGF LALFWDDLRS LCLFSYHRLR DLLLIVARIV
ELLGRRGWEA LKYWWNLLQY WSQELRNSAV SLLNATAIAV AEGTDRVIEI VQRIYRAILH
IPTRIRQGLE RLLL
ΔCT: amino acids 1-709
Cytoplasmic tail: amino acids 710-854
SEQ ID NO: 2-SARS-C0V-2 spike protein
MFVFLVLLPL VSSQCVNLTT RTQLPPAYTN SFTRGVYYPD KVFRSSVLHS TQDLFLPFFS
NVTWFHAIHV SGTNGTKRFD NPVLPFNDGV YFASTEKSNI IRGWIFGTTL DSKTQSLLIV
NNATNVVIKV CEFQFCNDPF LGVYHKNNKS WMESEFRVYS SANNCTFEYV SQPFLMDLEG
KQGNFKNLRE FVFKNIDGYF KIYSKHTPIN LVRDLPQGFS ALEPLVDLPI GINITRFQTL
LALHRSYLTP GDSSSGWTAG AAAYYVGYLQ PRTFLLKYNE NGTITDAVDC ALDPLSETKC
TLKSFTVEKG IYQTSNFRVQ PTESIVRFPN ITNLCPFGEV FNATRFASVY AWNRKRISNC
VADYSVLYNS ASFSTFKCYG VSPTKLNDLC FTNVYADSFV IRGDEVRQIA PGQTGKIADY
NYKLPDDFTG CVIAWNSNNL DSKVGGNYNY LYRLFRKSNL KPFERDISTE IYQAGSTPCN
GVEGFNCYFP LQSYGFQPTN GVGYQPYRVV VLSFELLHAP ATVCGPKKST NLVKNKCVNF
NFNGLTGTGV LTESNKKFLP FQQFGRDIAD TTDAVRDPQT LEILDITPCS FGGVSVITPG
TNTSNQVAVL YQGVNCTEVP VAIHADQLTP TWRVYSTGSN VFQTRAGCLI GAEHVNNSYE
CDIPIGAGIC ASYQTQTNSP RRARSVASQS IIAYTMSLGA ENSVAYSNNS IAIPTNFTIS
VTTEILPVSM TKTSVDCTMY ICGDSTECSN LLLQYGSFCT QLNRALTGIA VEQDKNTQEV
FAQVKQIYKT PPIKDFGGFN FSQILPDPSK PSKRSFIEDL LFNKVTLADA GFIKQYGDCL
GDIAARDLIC AQKFNGLTVL PPLLTDEMIA QYTSALLAGT ITSGWTFGAG AALQIPFAMQ
MAYRFNGIGV TQNVLYENQK LIANQFNSAI GKIQDSLSST ASALGKLQDV VNQNAQALNT
LVKQLSSNFG AISSVINDIL SRLDKVEAEV QIDRLITGRL QSLQTYVTQQ LIRAAEIRAS
ANLAATKMSE CVLGQSKRVD FCGKGYHLMS FPQSAPHGVV FLHVTYVPAQ EKNFTTAPAI
CHDGKAHFPR EGVFVSNGTH WFVTQRNFYE PQIITTDNTF VSGNCDVVIG IVNNTVYDPL
QPELDSFKEE LDKYFKNHTS PDVDLGDISG INASVVNIQK EIDRLNEVAK NLNESLIDLQ
ELGKYEQYIK WPWYIWLGFI AGLIAIVMVT IMLCCMTSCC SCLKGCCSCG SCCKFDEDDS
EPVLKGVKLH YT
ΔCT: amino acids 1-1233
Cytoplasmic tail: amino acids 1234-1272

What is claimed is:

1. A composition comprising:
a virus-like particle (VLP); and
a truncated form of a viral envelope protein displayed on the VLP, the truncated form comprising a deletion of at least a portion of a cytoplasmic tail region of the envelope protein.

2. The composition of claim 1, wherein:
the VLP comprises HIV-1 Gag; and
the truncated form of the viral envelope protein comprises HIV-1 envelope protein with at least a portion of the cytoplasmic tail deleted.

3. The composition of claim 1, further comprising an adjuvant.

4. A composition comprising:
a first polynucleotide encoding a VLP subunit protein;
a second polynucleotide encoding a viral envelope protein comprising a deletion of at least a portion of a cytoplasmic tail; and
a delivery vehicle attached to or encapsulating the first polynucleotide and the second polynucleotide together or separately.

5. The composition of claim 4, wherein the delivery vehicle comprises a lipid nanoparticle.

6. A composition comprising:
a first polynucleotide attached to or encapsulated by a first delivery vehicle, the first polynucleotide encoding a VLP subunit protein; and
a second polynucleotide attached to or encapsulated by a second delivery vehicle, the second polynucleotide encoding a viral envelope protein comprising a deletion of at least a portion of a cytoplasmic tail.

7. The composition of claim 6, wherein the first delivery vehicle, the second delivery vehicle, or both delivery vehicles comprises a lipid nanoparticle.

8. A method of preparing a VLP-based vaccine, the method comprising:
transfecting a cell with a first polynucleotide encoding a VLP subunit protein;
transfecting the cell with a second polynucleotide encoding a truncated viral envelope protein comprising a deletion of at least a portion of a cytoplasmic tail;
allowing the cell to express the first polynucleotide to produce VLP subunits;
allowing the cell to express the second polynucleotide to produce the truncated viral envelope protein; and
allowing the VLP subunits and the truncated viral envelope protein to assemble into a VLP in which the truncated viral envelope protein is displayed on the surface of the VLP.

9. The method of claim 8, wherein the cell is transfected in vitro.

10. The method of claim 8, wherein the cell is transfected in vivo.

11. The method of claim 8, wherein the cell is transfected with the first polynucleotide and the second polynucleotide simultaneously.

12. The method of claim 8, wherein the cell is transfected with the first polynucleotide and the second polynucleotide separately.

13. The method of claim 8, wherein transfecting the cell with the first polynucleotide or transfecting the cell with a second polynucleotide comprises using a lipid nanoparticle as a delivery vehicle.

14. A method of treating a subject having, or at risk of having, an infection by an enveloped virus, the method comprising:
administering to the subject a composition in an amount effective to treat infection by the enveloped virus, the composition comprising:
a virus-like particle (VLP); and
a truncated form of a viral envelope protein displayed on the VLP, the truncated form comprising a deletion of at least a portion of a cytoplasmic tail region of the envelope protein.

15. A method of treating a subject having, or at risk of having, an infection by an enveloped virus, the method comprising:
administering to the subject a composition in an amount effective to treat infection by the enveloped virus, the composition comprising:
a first polynucleotide encoding a VLP subunit protein;
a second polynucleotide encoding a viral envelope protein comprising a deletion of at least a portion of a cytoplasmic tail; and
a delivery vehicle attached to or encapsulating the first polynucleotide and the second polynucleotide.

16. The method of claim 15, wherein the delivery vehicle comprises a lipid nanoparticle.


SEQUENCE LISTING (2 sequences)

SEQ ID NO: 1 - protein, 854 aa, Human immunodeficiency virus 1
MKVKGIRKNYQHLWKWGIMLLGMLMICSAVENLWVTVYYGVPVWKEATTTLFCASDAKAYDTEVHNVWATHACVPTDPNPQEVVLENVTENFNMWKNNMVEQMHEDIISLWDQSLKPCVKLTPLCVTLNCTDLRNVTNINNSSEGMRGEIKNCSFNITTSIRDKVKKDYALFYRLDVVPIDNDNTSYRLINCNTSTITQACPKVSFEPIPIHYCTPAGFAILKCKDKKFNGTGPCKNVSTVQCTHGIRPVVSTQLLLNGSLAEEEVVIRSSNFTDNAKNIIVQLKESVEINCTRPNNNTRKSIHIGPGRAFYTTGDIIGDIRQAHCNISRTKWNNTLNQIATKLKEQFGNNKTIVFNQSSGGDPEIVMHSFNCGGEFFYCNSTQLFNSTWNFNGTWNLTQSNGTEGNDTITLPCRIKQIINMWQEVGKAMYAPPIRGQIRCSSNITGLILTRDGGNNHNNDTETFRPGGGDMRDNWRSELYKYKVVKIEPLGVAPTKAKRRVVQREKRAVGTIGAMFLGFLGAAGSTMGAASITLTVQARLLLSGIVQQQNNLLRAIEAQQHLLQLTVWGIKQLQARVLALERYLRDQQLLGIWGCSGKLICTTAVPWNASWSNKTLDMIWNNMTWMEWEREIDNYTGLIYTLIEESQNQQEKNEQELLELDKWASLWNWFDITNWLWYIKIFIMIVGGLIGLRIVFTVLSIVNRVRQGYSPLSFQTHLPAPRGPDRPEGIEEEGGDRDRDRSVRLVDGFLALFWDDLRSLCLFSYHRLRDLLLIVARIVELLGRRGWEALKYWWNLLQYWSQELRNSAVSLLNATAIAVAEGTDRVIEIVQRIYRAILHIPTRIRQGLERLLL

SEQ ID NO: 2 - protein, 1272 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT